(19) United States
(12) Patent Application Publication
(10) Pub. No.: US 2008/0086101 A1
(43) Pub. Date: Apr. 10, 2008

(54) Ophthalmic insert

(76) Inventor: David Freilich, Englewood, NJ (US)

Correspondence Address: EDELL, SHAPIRO & FINNAN, LLC, 1901 RESEARCH BOULEVARD, SUITE 400, ROCKVILLE, MD, 20850, US

(21) Appl. No.: 11/641,903

(22) Filed: Dec. 20, 2006

Related U.S. Application Data
(60) Provisional application No. 60/840,039, filed on Aug. 25, 2006.

(51) Int. Cl. A61M 35/00

(52) U.S. Cl. 604/294

(57) ABSTRACT

A medical device for delivering medication to an eye includes a stent having a proximal end and a distal end, the proximal end includes a collarette configured to be secured against a punctum of the nasal lacrimal system, and the distal end includes an expandable pouch for storing the medication to be delivered to the eye. The stent has a length substantially equivalent to a length of a canaliculus that is joined to the punctum such that, when implanted, the expandable pouch of the medical device is disposed in the nasal lacrimal sac of the patient and the medication is thereafter released through an opening in the collarette. A mechanical pumping mechanism in the expandable pouch and/or a membrane covering the opening in the collarette may be provided to better control release of the fluid. The stent may also include anchoring pegs disposed on an exterior surface thereof that, when the device is implanted, contact an interior surface of the canaliculus. An inserter tool is also described.

This Application claims the benefit of U.S. Provisional application No. 60/840,039, filed Aug. 25, 2006, which is incorporated herein by reference in its entirety.

### BACKGROUND OF THE INVENTION

The vast majority of eye medication is delivered via liquid drops by a conventional eye dropper. While this delivery mechanism has proven effective, it also has several drawbacks. For example, much of the medication runs off the eye before it can be absorbed or penetrate into the eye. Further, the medication is not applied uniformly over time in that there is an initial higher concentration of drug immediately upon application, as compared to subsequent time periods. Finally, patients often forget to use their medication, or are incapable of properly administering the drops for themselves. In other words, it is not uncommon for patients to fail to medicate themselves sufficiently, or at the correct times.

U.S. Pat. No. 6,196,993 to Cohan et al. describes an ophthalmic device that purportedly overcomes the failings of the conventional medication delivery methods noted above. Cohan et al. propose a conventional punctal occluder that is reconfigured to include a reservoir for storing and releasing medication over time. The punctual occluder is preferably positioned in the upper lacrimal drainage system, namely the lacrimal punctum and canaliculus. In order to increase the volume of the reservoir, a reservoir extension may also be provided as shown in FIG. 5 of the '993 patent. The extension is a balloon-like component that extends only into the canaliculus.

Although the device described by Cohan et al. appears to provide some advantages over the prior art eye medication delivery mechanisms, there is nevertheless a continuing need for improvements in ocular medication delivery.

### SUMMARY OF THE INVENTION

An embodiment of the present invention provides a flexible stent or tube that is inserted through the lacrimal punctum and canaliculus and includes, on one end, an expandable pouch that rests in the nasal lacrimal sac and, on the other end, a collarette having a passageway therethrough that is in fluid communication with the expandable pouch. Optionally, anchoring pegs may be provided on the stent or tube that help to secure the device in the canaliculus. The expandable pouch is filled with medication, and the medication is thereafter permitted to flow naturally (e.g., through capillary action), through digital pressure applied by the patient to the nasal lacrimal sac, and/or with the assistance of a miniature pump back out through the passageway and onto the eye. Because the expandable pouch is disposed in the relatively large cavity of the nasal lacrimal sac, it is possible to fill the expandable pouch with enough medication such that therapy from a single filling of the pouch may last weeks, or perhaps months.

Also provided in accordance with an embodiment of the present invention is a unique inserter tool for inserting the medical device. In one embodiment, the stent is fitted with a one-way valve through which the inserter tool is passed into the interior of the stent so that the stent can be properly positioned in the canaliculus and the expandable pouch in the nasal lacrimal sac. The inserter tool is then withdrawn. A filling syringe having a tip that is configured to fit into the opening of the collarette (or into an opening of the inserter tool before the inserter tool is withdrawn) is preferably provided and is used to fill or refill the expandable pouch as necessary.

These and other features of the present invention, along with their attendant advantages, will be even more appreciated by those skilled in the art upon a reading of the following detailed description in conjunction with the accompanying several drawing figures.

### BRIEF DESCRIPTION OF THE DRAWINGS

FIG. 1 depicts a general view of the anatomy of an eye, along with an embodiment of an ophthalmic insert in accordance with the present invention;

FIG. 2 depicts the structure of a collarette in accordance with an embodiment of the present invention;

FIG. 3 depicts an exemplary ophthalmic insert in accordance with an embodiment of the present invention;

FIG. 4 depicts an inserter tool for inserting and implanting the ophthalmic insert in a patient in accordance with an embodiment of the present invention; and

FIG. 5 depicts an exemplary syringe used for filling the ophthalmic insert with medication in accordance with an embodiment of the present invention.

### DETAILED DESCRIPTION

There are many different pathologies and conditions that require the use of ophthalmic medications in the form of eye drops. Some of the most common conditions include infections, glaucoma, and “dry eye syndrome.” There are, correspondingly, many different types of medications for these conditions that require frequent administration. Often, however, patient compliance is problematic, so much so that it can render medication ineffective. Also, for various technical reasons, many patients, especially the elderly, have difficulty administering eye drops.

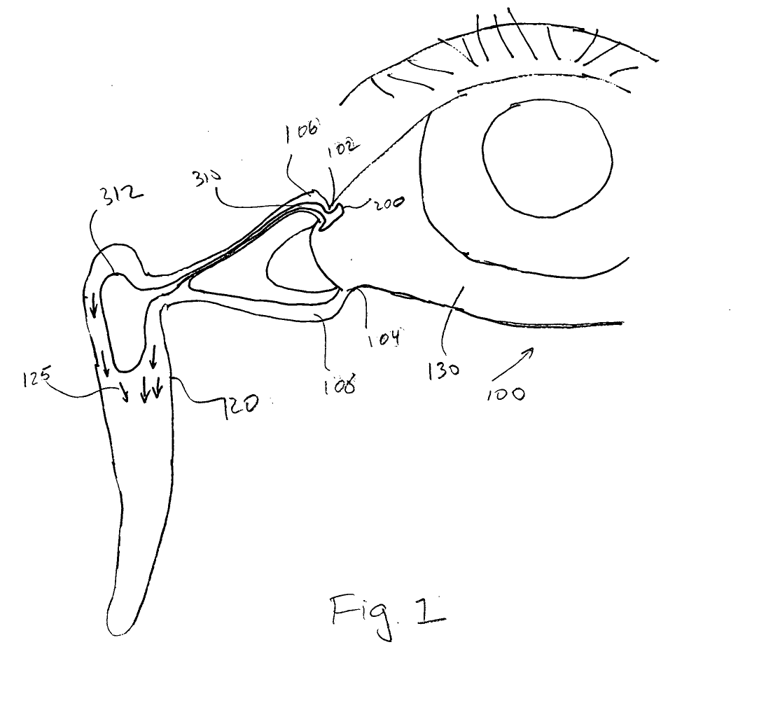

FIG. 1 depicts a general view of the anatomy of an eye 100, including upper and lower puncta 102, 104 each forming an opening to a canaliculus 106, 108 that respectively lead to the nasal lacrimal sac 120. As is well known, fluid (e.g., tears) on the eye may be drawn through puncta 102, 104 and drain into the nasal lacrimal sac 120 as shown by reference numeral 125.

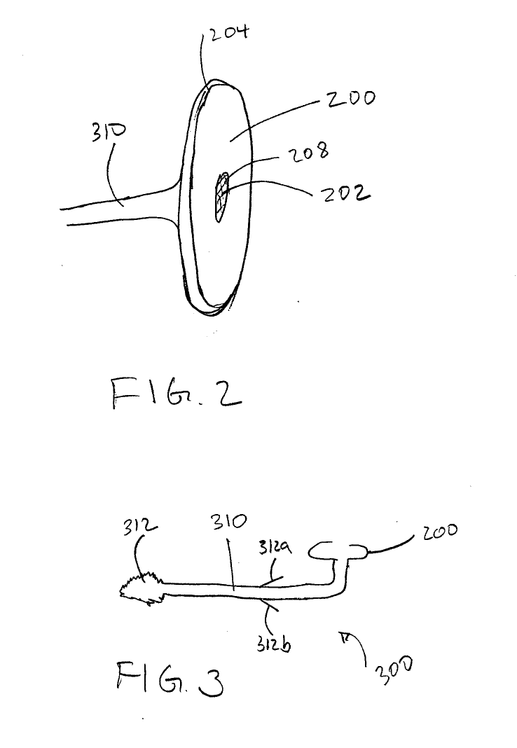

Also shown in FIG. 1, and also now with reference to FIGS. 2 and 3, is an embodiment of an ophthalmic insert in accordance with the present invention that comprises an implantable device 300 that seeks to deliver ophthalmic medication directly onto the ocular surface 130 in a time-released manner. This device may dramatically improve patient compliance, assure proper drug delivery and dosing, and may have the added benefit of reducing cost while improving patient comfort.

More specifically, there is provided a stent or tube 310 (hereafter referred to as a “stent”) with an expandable pouch 312 at its distal end and a collarette 200 at its proximal end. The expandable pouch is used to store ophthalmic medication in liquid or other form (not shown) that can be secreted or pumped onto the surface of the eye.

Stent 310 is preferably made of silicone or other inorganic, flexible material. As shown, stent 312 is inserted through the lacrimal punctum 102 and canaliculus 106 such that the expandable pouch is disposed in the nasal lacrimal sac 120. Collarette 200 includes a flange portion 204 that rests against an exterior portion of punctum 102. Just posterior to collarette 200 are (optionally) small anchoring pegs 312a, 312b that flex against the interior wall of the canaliculus after insertion to further anchor the stent 310 in place within the canaliculus. Collarette 200 further includes an opening 202 that may be covered by a suitable membrane 208 and through which medication is slowly released onto the ocular surface.

As a result of the structure of device 300, there is provided an open channel or passageway that extends from the opening 202 of collarette 200, through the entire length of the stent 310 and into the expandable pouch 312 at the distal end thereof. Ocular medication is stored within pouch 312. In one possible implementation, expandable pouch 312 may also include a micro pump (or other nanotechnology apparatus) to enhance control over the delivery of the stored medication. Because the patient's normal tears (e.g., element 125) must drain properly, expandable pouch 312 is preferably not expanded to entirely fill the volume of the nasal lacrimal sac 120, but is nonetheless sufficiently filled with medication to provide sustained release of medication.

The use of expandable pouch 312, which rests in the nasal lacrimal sac 120, is a critical and necessary improvement to the ophthalmic device disclosed in U.S. Pat. No. 6,196,993 to Cohan et al. The device disclosed herein is a much improved implantable medical device that allows a much greater volume of medication to be stored for sustained release thereof. As a result, filling of the implant is less frequent, thereby resulting in fewer doctor visits and lower cost. The implantable device 300 may also remain in place for a sustained period of time as medication can be easily refilled, as discussed further below.

The device 300 can be placed into any punctum and canaliculus in a non-operative procedure without anesthesia. The procedure may be performed, for example, in a physician's office thereby avoiding the need for costly hospital and operating rooms.

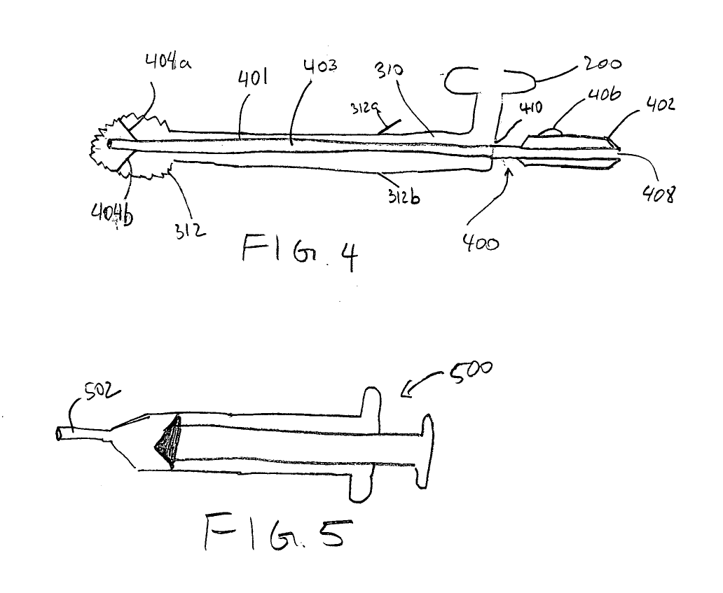

In a preferred embodiment, there is provided an inserter tool 400 as shown in FIG. 4. Inserter tool 400 is comprised of, e.g., a metal probe 401 with an open core 403 that runs the length of the tool. Probe 401 rests within the open channel inside stent 310 and extends substantially the entire length of stent 310. The distal end of the probe 401 may include small anchoring pegs 404a, 404b that can push against a distal end of stent 310 or against an interior wall of the expandable pouch 312, as shown. The implantable device 300 is preferably passed through punctum 102, for example. Once the device 300 is in place and the expandable pouch is securely resting in the nasal lacrimal sac, a button 406 on handle 402 can be activated to disconnect anchoring pegs 404a, 404b from the device. Once released, probe 401 is gently retracted approximately three quarters out of stent 310 and a syringe 500 (FIG. 5) having a flexible tip 502 is attached to an opening 408 at the proximal end of probe 401. The ophthalmic medication is then injected from the syringe through the channel passing through the stent thus filling expandable pouch 312 within the nasal lacrimal sac 120. After filling the pouch with medication, the inserter tool is removed and disposed of or recycled.

In the embodiment shown in FIG. 4, medical device 300 includes a one-way valve 410 through which probe 401 is passed. This allows an appropriate size hole in device 300 to accommodate probe 401. That is, opening 202 in collarette 200 may be too small for this purpose, and in any event, may be covered by a membrane that should not be punctured. Thus, once the filling of expandable pouch 312 is complete and the probe 401 is fully removed, collarette 200 is pushed towards punctum and inserted therein. Of course, if there is no membrane or the membrane is easily replaceable, then to the extent probe 401 fits through opening 202, there may be no need for one-way valve 410.

The rate of depletion of the stored medication will depend on several factors specific to the case being treated, but eventually the medication will likely be fully depleted. However, the device 300 can be refilled without removing it from punctum 102, canaliculus 106 or nasal lacrimal sac 120. This is accomplished by again using syringe 500 with flexible tip 502 that will fit into the opening 202. Of course, again, if there is a non-removable membrane covering opening 202, then collarette 200 is preferably pulled out of the punctum 102 sufficient to expose one-way valve 410 such that re-filling can proceed in the manner described above.

In summary, this refillable, sustained drug-release ophthalmic implant is a substantial improvement over known implants for several reasons:

The expandable pouch provides for increased storage of ophthalmic medication within the lacrimal sac. This advancement reduces the number of visits to the doctor's office otherwise required to refill the device.

By allowing the bulk of a normal course of medication to be stored within the nasal lacrimal sac, less pressure is placed onto the canaliculus making the device more comfortable for the patient.

In addition, the medical device of the present invention remains securely implanted in a patient due to the fact that it runs the length of the canaliculus and is secured on one end in punctum 102 by collarette 200 and on the other end in the nasal lacrimal sac by the expandable pouch 312. The secure nature of this implantable device is far superior to conventional punctal plugs that may easily or inadvertently fall out.

The insert tool allows for a simple procedure to be conducted within a physician's office without the need for anesthesia. This relatively simple procedure is comfortable for the patient and avoids the need for any surgical procedures, thereby reducing costs.

The specifically configured syringe allows for simple refilling with any ophthalmic medication without the need to remove the entirety of the implantable device.

Finally, the implant can be easily removed if needed, again in the physician's office without anesthesia and without damaging the nasal lacrimal sac, canaliculus or punctum.

The foregoing disclosure of the preferred embodiments of the present invention has been presented for purposes of illustration and description. It is not intended to be exhaustive or to limit the invention to the precise forms disclosed. Many variations and modifications of the embodiments described herein will be apparent to one of ordinary skill in the art in light of the above disclosure. The scope of the invention is to be defined only by the claims appended hereto, and by their equivalents.

Further, in describing representative embodiments of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily appreciate that the sequences may be varied and still remain within the spirit and scope of the present invention.

What is claimed is:

1. An apparatus for delivering a sustained release of ocular medication to an eye, comprising:
a stent having a proximal end and a distal end, the proximal end including a collarette configured to be secured against a punctum of the nasal lacrimal system, and the distal end including an expandable pouch for storing the medication to be delivered to the eye, the stent having a length substantially equivalent to a length of a canaliculus that is joined to the punctum.

2. The apparatus of claim 1, further comprising a pumping mechanism for controlling a sustained release of the fluid.

3. The apparatus of claim 1, wherein the medication comprises liquid medication.

4. The apparatus of claim 1, wherein, when implanted, the expandable pouch is disposed in the nasal lacrimal sac.

5. The apparatus of claim 1, wherein a fluid passageway is defined by the expandable pouch, the stent, and an opening in the collarette, which opening allows the medication to reach a surface of the eye.

6. The apparatus of claim 1, being comprised of a flexible material.

7. The apparatus of claim 6, wherein the flexible material comprises silicone.

8. The apparatus of claim 1, further comprising anchoring pegs disposed on an exterior surface of the stent posterior to the collarette.

9. The apparatus of claim 1, further comprising a membrane covering a portion of the collarette to control delivery of the medication to the eye.

10. The apparatus of claim 1, wherein the stent includes a one-way valve through which the medication can be introduced into the stent and thereby also into the expandable pouch.

11. An ophthalmic insert for sustained release of medication to an eye, comprising:
a flexible tube having an interior passageway;
a collarette having a passageway coaxially arranged with the passageway of the flexible tube and attached to a first end of the flexible tube; and
an expandable pouch, configured to store the medication, attached to a second end of the flexible tube, the flexible tube having a length substantially equivalent to a length of a canaliculus that is joined to the punctum such that, when the flexible tube, collarette and expandable pouch are implanted in a patient, the expandable pouch is disposed in the nasal lacrimal sac of the patient.

12. The ophthalmic insert of claim 1, further comprising a pumping mechanism for controlling a sustained release of the medication.

13. The ophthalmic insert of claim 11, wherein the ophthalmic insert is comprised of a flexible material.

14. The ophthalmic insert of claim 13, wherein the flexible material comprises silicone.

15. The ophthalmic insert of claim 11, further comprising anchoring pegs disposed on an exterior surface of the flexible tube.

16. The ophthalmic insert of claim 11, further comprising a membrane covering a portion of the collarette to control delivery of the medication to the eye.

17. The ophthalmic insert of claim 11, wherein the flexible tube includes a one-way valve through which the medication can be introduced into the flexible tube and thereby also into the expandable pouch.

18. A medical apparatus for delivering medication to an eye of a patient, comprising:
a stent having a proximal end and a distal end, the proximal end including a collarette configured to be secured against a punctum of the nasal lacrimal system, and the distal end including an expandable pouch for storing the medication to be delivered to the eye, the stent having a length substantially equivalent to a length of a canaliculus that is joined to the punctum; and
an inserter tool comprising a probe and anchoring pegs configured to push against at least one of an interior of the distal end of the stent and an interior of the expandable pouch.

19. The medical apparatus of claim 18, wherein the collarette defines an opening that is covered by a membrane for controlling sustained release of medication to the eye.

20. The medical apparatus of claim 18, wherein the stent comprises anchoring pegs disposed on an exterior surface thereof.
